(19) United States
(12) Patent Application Publication
(10) Pub. No.: US 2025/0367399 A1
(43) Pub. Date: Dec. 4, 2025

(54) MEDICAL HUMIDIFIER OPERABLE IN ALTERNATING CURRENT AND DIRECT CURRENT

(71) Applicant: Seoil Pacific Corp., Seoul (KR)

(72) Inventor: Kye Cheol KIM, Goyang-si (KR)

(21) Appl. No.: 19/222,033

(22) Filed: May 29, 2025

(30) Foreign Application Priority Data
Jun. 3, 2024 (KR) 10-2024-0072161

(51) Int. Cl. A61M 16/10; A61M 16/00; A61M 16/08; G16H 40/63

(52) U.S. Cl. CPC A61M 16/109; A61M 16/0003; A61M 16/022; A61M 16/0875; A61M 16/1095; G16H 40/63; A61M 2202/0007; A61M 2202/02; A61M 2205/3327; A61M 2205/3368; A61M 2205/3653; A61M 2205/502

(57) ABSTRACT

The present embodiment is a medical humidifier comprising: a water tank mountable to the medical humidifier, having an air inlet through which air is introduced and an air outlet through which humidified air is discharged; a heating unit that heats the water tank; and a housing on which the water tank is mounted. The housing includes: an alternating current inlet and a direct current inlet, a conversion circuit mounted inside that converts the alternating current supplied to the alternating current inlet into a first direct current voltage and outputs it, and a control unit. The control unit controls the heating unit to heat, wherein the heating unit is heated by either the first direct current voltage or a second direct current voltage provided to the direct current inlet.

### CROSS-REFERENCE TO RELATED APPLICATIONS

This application claims priority to and the benefit of Korean Patent Application No. 10-2024-0072161 filed on Jun. 3, 2024, the entire disclosure of which is incorporated herein by reference for all purposes.

### BACKGROUND

### Technical Field

The present disclosure generally relates to a medical humidifier operable in alternating current and direct current.

### Description of Related Art

A typical ultrasonic humidifier stores water in a water tank and vibrates a vibration plate located in the water tank to forcibly vaporize water molecules. The water vapor is released to the outside through the outlet of the humidifier, generally humidifying the indoor environment. Conventional humidifiers receive power to provide power for vibrating the vibration plate, and it is more common to control the amount of water vapor discharged by adjusting the vibration intensity and/or vibration frequency of the vibration plate using a handle or similar means. Prior art for ultrasonic humidifiers includes Korean Patent Publication No. 2020-0095586, among others.

Another type of humidifier is an evaporative humidifier, wherein water in the water tank humidifies the indoor space by evaporating moisture from an evaporation source such as cloth or non-woven fabric that has one end soaked in water and is wetted by capillary action. Prior art for evaporative humidifiers includes Korean Patent Registration No. 10-2052177, among others.

Conventional humidifiers use only alternating current power attached to the wall to provide humidified air to users, which presents difficulties when it is necessary to provide humidified air while moving a patient from one location to another in an emergency situation, as they cannot provide humidified air during transport.

One of the problems to be solved by the present embodiment is to address the difficulty of the prior art described above.

### SUMMARY

The present embodiment is a medical humidifier comprising: a water tank mountable to the medical humidifier, having an air inlet through which air is introduced and an air outlet through which humidified air is discharged; a heating unit that heats the water tank; and a housing on which the water tank is mounted, wherein the housing includes: an alternating current inlet and a direct current inlet, a conversion circuit mounted inside that converts the alternating current supplied to the alternating current inlet into a first direct current voltage and outputs the first direct current voltage, and a control unit, and wherein the control unit controls the heating unit to heat, wherein the heating unit is heated by either the first direct current voltage or a second direct current voltage provided to the direct current inlet.

According to one aspect of the present embodiment, the first direct current voltage and the second direct current voltage are provided to a switch unit including one or more switches, and the control unit controls the heating unit by controlling the switch unit.

According to one aspect of the present embodiment, the medical humidifier heats the heating unit with the second direct current when the alternating current is not supplied to the alternating current inlet and the second direct current is supplied to the direct current inlet.

According to one aspect of the present embodiment, the heating unit includes a first heating line to which the first direct current is applied and a first plate on which the first heating line is formed, and a second heating line to which the second direct current is applied and a second plate on which the second heating line is formed, wherein the first plate and the second plate are formed by being stacked to be electrically insulated from each other.

According to one aspect of the present embodiment, the first plate heats the water tank with higher power compared to the second plate.

According to one aspect of the present embodiment, the control unit controls the heating unit in a PWM (Pulse Width Modulation) manner.

According to one aspect of the present embodiment, the medical humidifier further includes a guiding hose coupled to the air outlet to guide the humidified air to a user.

According to one aspect of the present embodiment, the medical humidifier further includes a temperature sensor coupled to the end of the guiding hose to detect the temperature of the humidified air, and the housing further includes a temperature sensor connector to which the temperature sensor is connected.

According to one aspect of the present embodiment, the guiding hose is equipped with a heating wire that heats the guiding hose, and the housing further includes a guiding hose heating connector that provides heating power to the heating wire.

According to one aspect of the present embodiment, the housing further includes a display that displays the current temperature of the humidified air.

### BRIEF DESCRIPTION OF THE DRAWINGS

FIG. 1 is a perspective view illustrating an overview of a medical humidifier according to the present embodiment.

FIG. 2 is a view illustrating one side of the housing of the medical humidifier.

FIG. 3 is a view illustrating another side of the housing of the medical humidifier.

FIG. 4 is a view illustrating the use state of the medical humidifier of the present embodiment.

FIG. 5A is a view exemplifying the structure of the first plate, and FIG. 5B is a view exemplifying the structure of the second plate.

FIG. 6 is a block diagram illustrating an overview of the present embodiment.

### DETAILED DESCRIPTION

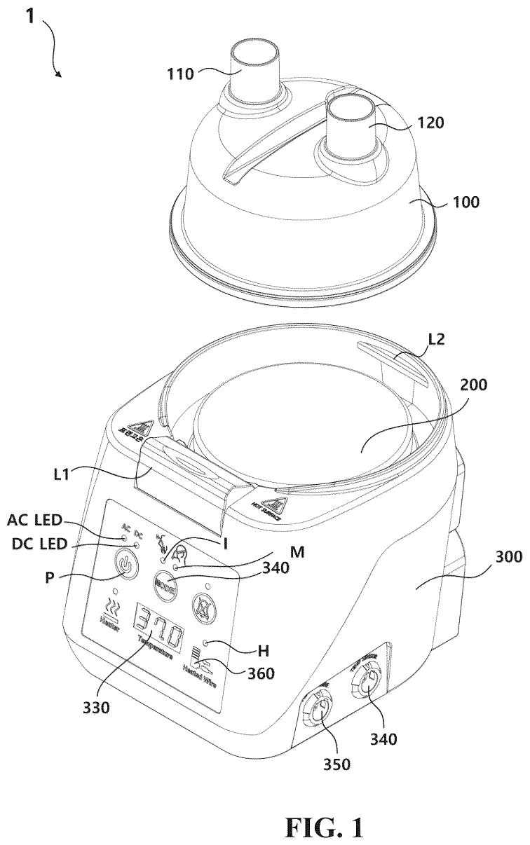

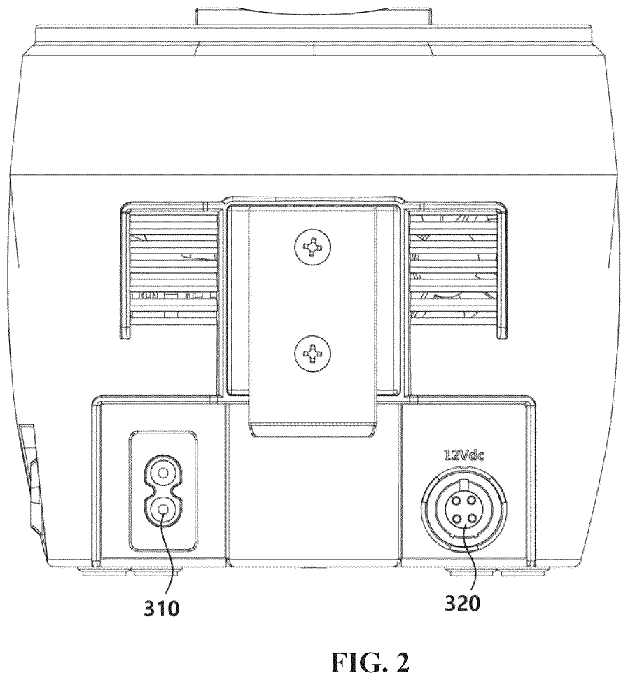

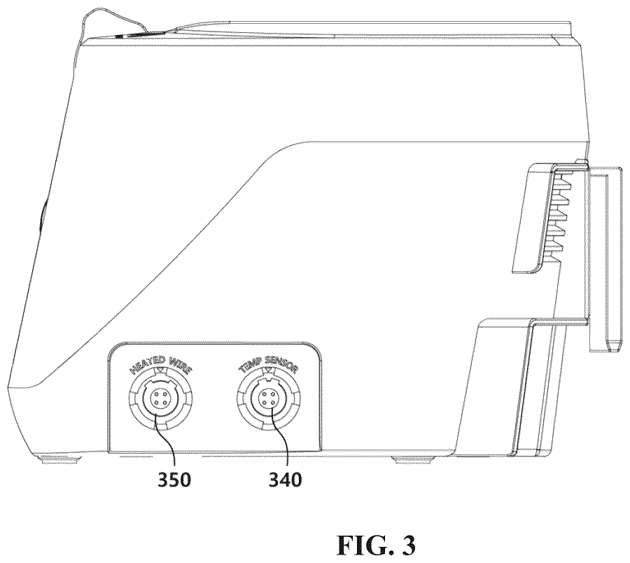

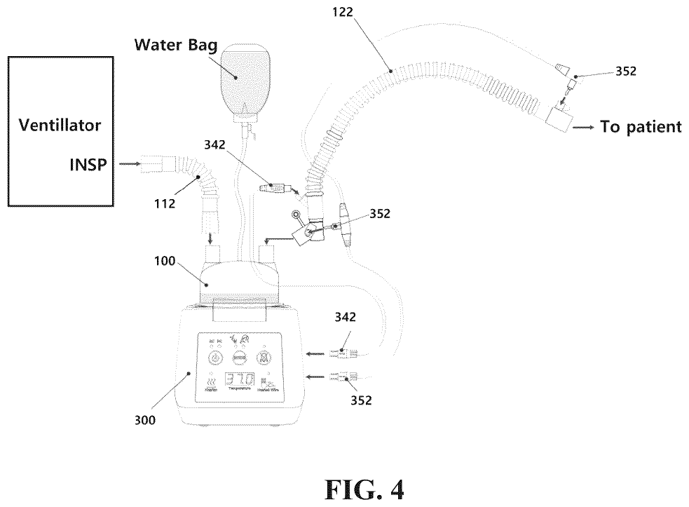

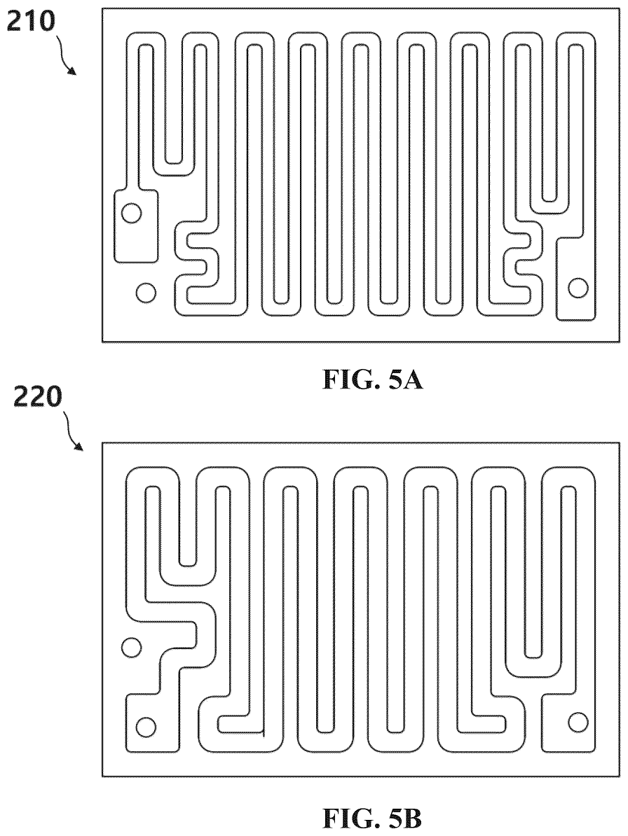

Hereinafter, the present embodiment will be described with reference to the accompanying drawings. FIG. 1 is a perspective view illustrating an overview of a medical humidifier 1 according to the present embodiment, FIG. 2 is a view illustrating one side of the medical humidifier housing 300, FIG. 3 is a view illustrating another side of the medical humidifier housing 300, and FIG. 4 is a view illustrating the use state of the medical humidifier of the present embodiment. Referring to FIGS. 1 to 4, the medical humidifier 1 according to the present embodiment includes a mountable water tank 100, a heating unit 200 that heats the water tank 100, and a housing 300 on which the water tank 100 is mounted. The housing 300 includes: an alternating current inlet 310, a direct current inlet 320, and a temperature display 330, and internally mounts an SMPS 510, (see FIG. 5) that converts alternating current applied to the alternating current inlet 310 into a first direct current and a control unit 500. The control unit 500 controls the heating unit 200 to heat with either the second direct current provided to the direct current inlet 320 or the first direct current.

The water tank 100 stores water and is heated by contact with the heating unit 200. In one embodiment, the surface where the water tank 100 and the heating unit 200 contact each other may have corresponding shapes. In the illustrated example, the water tank 100 and the heating unit 200 are shown to directly contact each other with a circular cross-section. However, in an embodiment not shown, the water tank and the heating unit may contact each other with a square, rectangular, or other cross-sectional shape.

The water tank 100 includes an air inlet 110 through which air is introduced and an air outlet 120 through which humidified air formed by heating the water located in the water tank 100 is discharged. In one embodiment, the air inlet 110 may be connected to a ventilator that provides air for the user to inhale through a hose, allowing air to enter the inside of the water tank. Also, the air outlet 120 may be connected to a hose 122 to provide humidified air to the user.

The housing 300 is formed with a fixing ledge L2 and a push member L1 for mounting and fixing the water tank 100. First, one end of the water tank 100 is inserted between the fixing ledge L2 and the heating unit 200. The water tank 100 is positioned considering the location of the air inlet 110 and air outlet 120 formed on the water tank 100 and the position of the patient to whom the humidified air is provided. Then, the end of the push member L1 is pressed and the opposite end of the water tank 100 is inserted so that the bottom surface of the water tank 100 directly contacts the heating unit 200. The push member L1 pushes with spring elasticity so that the water tank 100 does not separate from and remains in contact with the heating unit 200 to be heated.

In one embodiment, the medical humidifier 1 may further include a limit switch 250. The limit switch 250 is a switch that is connected or blocked when the water tank is mounted, from which the control unit 500 can heat the heating unit 200 only when the water tank is mounted.

When the water tank 100 is not mounted, the heating unit 200 is exposed in the housing 300. The heating unit 200 may be formed by stacking the first plate 210 and the second plate 220 to be insulated from each other. As an example, the heating unit 200 may include a heating unit cover that covers the stacked first plate and second plate.

FIG. 5A is a view exemplifying the structure of the first plate 210, and FIG. 5B is a view exemplifying the structure of the second plate 220. The first plate 210 exemplified in FIG. 5A is a heater with a conductive pattern formed on a mica plate. In one embodiment, the conductive pattern is a conductor such as nickel or chromium, which generates Joule heat when current is provided and heats the water tank 100. As an example, the first plate 210 is provided with direct current 24V converted from AC voltage and heats the water tank 100 with 140 W of power.

The second plate 220 exemplified in FIG. 5B is a heater with a conductive pattern formed on a mica plate. In one embodiment, the conductive pattern, like the first plate 210, is a conductor such as nickel or chromium, which is heated by Joule heat when current is provided and heats the water tank 100. As an example, the second plate 220 is provided with direct current 12V voltage from the direct current inlet 320 and heats the water tank 100 with 70 W of power.

When the medical humidifier 1 of the present embodiment is operated with alternating current power, the control unit 500 drives the first plate 210 with the first direct current converted from alternating current power. However, when the medical humidifier 1 is operated with direct current power, the control unit 500 drives the second plate 220 with the second direct current, which is the direct current voltage provided to the direct current inlet.

When driving the second plate 220 with the second direct current, since drive power is provided from a battery or similar source, power consumption needs to be reduced. Therefore, the second plate 220 is designed to be driven with lower power compared to the power driving the first plate 210.

On the other hand, when driving the first plate 210 with the first direct current, power consumption is more flexible compared to situations where power is supplied from a battery or similar source. Therefore, the first plate can be driven with higher power compared to the second plate 220. As an example, the first plate 210 may have the resistance value of the heating lines formed on the first plate 210 and the second plate 220 set so that the first plate 210 can heat the water tank 100 with more than twice the power compared to the second plate 220.

The humidifier of the present embodiment can operate with power using direct current voltage provided through a battery. Therefore, it provides the advantage of being able to provide humidified air even during patient transport.

The housing 300 includes an alternating current inlet 310, a direct current inlet 320, and a temperature display 330. An alternating current voltage of 110V to 240V provided from a wall-mounted alternating current power source can be provided to the alternating current inlet 310. Direct current 12V power can be provided through the direct current inlet 320, as exemplified in FIGS. 1 and 2. Therefore, present embodiment provides the advantage of being able to receive direct current voltage from a vehicle battery during patient transport and continuously provide humidified air to the patient. In another example not shown, direct current voltages such as 18V, 24V, etc. can be provided to the direct current inlet.

The housing 300 is formed with a power button P, and when the medical humidifier 1 is operated by pressing the power button P, either the alternating current LED AC LED or the direct current LED DC LED lights up depending on the power source driving the medical humidifier 1. Therefore, the user can easily identify whether the humidifier's driving power is alternating current or direct current.

The housing 300 may include a mode button 340 that controls the temperature of the air provided. In one embodiment, the medical humidifier 1 of the present embodiment may provide humidified air through a hose that has one end connected to the air outlet 120 and the other end connected to a hose that is intubated into the patient's airway. When the mode for providing humidified air through the intubated hose is selected by pressing the mode button 340, the intubation mode LED I lights up. When providing humidified air through an intubated hose, the humidifier 1 forms and provides humidified air at a temperature of 35 to 39 degrees in Celsius, similar to the human body temperature, to the patient.

In another embodiment, the medical humidifier 1 of the present embodiment may provide humidified air through a hose that has one end connected to the air outlet 120 and the other end connected to the patient's mask. When the case of providing humidified air through a hose connected to a mask is selected by pressing the mode button 340, the mask mode LED M lights up. When providing humidified air through a hose connected to a mask, it is preferable to maintain an air temperature of 30 to 35 degrees in Celsius.

The housing 300 includes a temperature sensor connector 340 that connects with a temperature sensor 352 located on the guiding hose 122 that guides the humidified air. The temperature sensor 352 detects the temperature of the humidified air, and the control unit 500 displays the temperature of the humidified air provided to the user through the display 330 from the temperature sensor. The temperature of the humidified air detected by the control unit 500 may be displayed through the temperature display 330.

The housing 300 is formed with a hose heating indicator H and a hose heating wire connector 350 that connects with a hose heating wire 342 located on the hose. The temperature of the humidified air may decrease as it moves through the hose, and in such cases, water vapor may condense inside the hose. To prevent condensation, when the connection between the hose heating wire 342 and heating wire connector 350 is established, the hose heating wire receives heating power from the hose heating wire connector 350 of the housing 300 and heats the hose. When heating the hose by connecting the hose heating wire 342, the hose heating indicator H may light up to notify the user of hose heating.

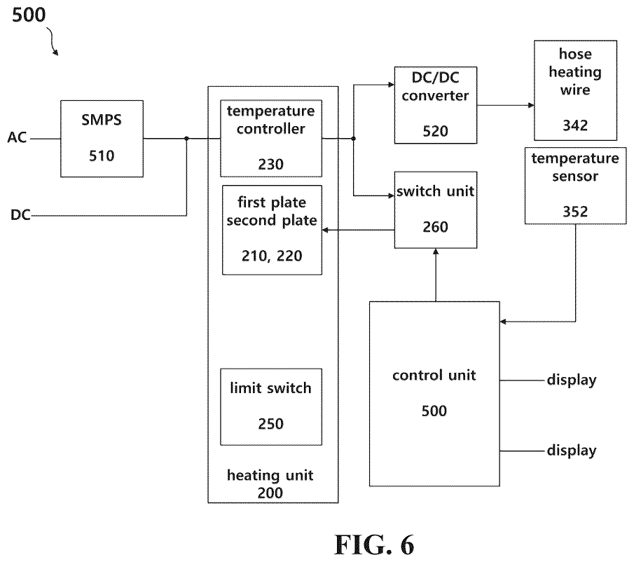

FIG. 6 is a block diagram exemplifying the electrical connection relationship of the medical humidifier 1 according to the present embodiment. Referring to FIGS. 1 to 6, when an alternating current voltage is applied to the SMPS switching mode power supply, 510 through the alternating current inlet 310, the SMPS 510 forms and outputs the desired first direct current voltage. In one embodiment, the first direct current voltage output by the SMPS 510 may be 24V and may be a higher voltage compared to the second direct current voltage provided through the direct current inlet 320.

The first direct current or the second direct current provided through the direct current inlet 320 is provided to the first plate 210 and the second plate 220 of the heating unit 200 through the temperature controller 230 and a switch unit 260 that includes switches controlled by the control signal of the control unit 500.

As shown in FIG. 6, when alternating current is not applied to the alternating current inlet 310 and the second direct current is applied to the direct current inlet 320, the second direct current is provided to the heating unit 200 through the temperature controller 230 and the switch unit 260 to heat the first plate 210 and/or the second plate 220. In one embodiment, the temperature controller 230 may be a thermostat, which blocks the provided first direct current and/or second direct current power when the first plate 210 or the second plate 220 is heated above the set temperature.

The control unit 500 detects the temperature of the humidified air provided to the user from the temperature sensor 352 attached to the guiding hose 122 and controls the temperature of the heating unit 200 by controlling the connection and/or blocking of the switches included in the switch unit 260. In one embodiment, the control unit 500 may control the temperature by controlling the connection and/or blocking of the switches included in the switch unit 260 in a PWM (Pulse Width Modulation manner.

The direct current voltage provided through the temperature controller 230 is applied to the DC/DC converter 520, and the hose heating wire 342 may be heated with the voltage output from the DC/DC converter 520. As described above, the control unit 500 can determine the temperature of the humidified air provided to the user through the guiding hose 122 from the temperature sensor 352, and from this, control the temperature at which the heating unit 200 heats the water tank. By controlling as described above, heated and humidified air at a temperature suitable for the patient can be provided.

The present invention has been described with reference to the embodiments illustrated in the drawings to help understand the invention, but these are exemplary embodiments for implementation and are merely examples. Those skilled in the art will understand that various modifications and equivalent other embodiments are possible from this. Therefore, the true technical scope of protection of the present invention should be determined by the appended claims.

What is claimed is:

1. A medical humidifier, comprising:
a water tank mountable to the medical humidifier, having an air inlet through which air is introduced and an air outlet through which humidified air is discharged;
a heating unit that heats the water tank; and
a housing on which the water tank is mounted,
wherein the housing includes:
an alternating current inlet and a direct current inlet,
a conversion circuit mounted inside the housing, the conversion unit converts alternating current applied to the alternating current inlet into a first direct current voltage and outputs the first direct current voltage,
a control unit mounted inside the housing, and
wherein the control unit controls the heating unit to heat, and
wherein the heating unit is heated by either the first direct current voltage or a second direct current voltage provided to the direct current inlet.

2. The medical humidifier of claim 1,
wherein the first direct current voltage and the second direct current voltage are provided to a switch unit including one or more switches, and
wherein the control unit controls the heating unit by controlling the switch unit.

3. The medical humidifier of claim 1,
wherein the medical humidifier heats the heating unit with the second direct current when the alternating current is not applied to the alternating current inlet and the second direct current is applied to the direct current inlet.

4. The medical humidifier of claim 1,
wherein the heating unit includes:
a first heating line to which the first direct current is applied and a first plate on which the first heating line is formed, and
a second heating line to which the second direct current is applied and a second plate on which the second heating line is formed,
wherein the first plate and the second plate are formed by being stacked to be electrically insulated from each other.

5. The medical humidifier of claim 4,
wherein the first plate heats the water tank with higher power compared to the second plate.

6. The medical humidifier of claim 1,
wherein the control unit controls the heating unit in a PWM (Pulse Width Modulation) manner.

7. The medical humidifier of claim 1,
wherein the medical humidifier further includes a guiding hose coupled to the air outlet to guide the humidified air to a user.

8. The medical humidifier of claim 7,
wherein the medical humidifier further includes a temperature sensor coupled to the end of the guiding hose to detect the temperature of the humidified air, and
wherein the housing further includes a temperature sensor connector to which the temperature sensor is connected.

9. The medical humidifier of claim 7,
wherein the guiding hose is equipped with a heating wire that heats the guiding hose, and
wherein the housing further includes a guiding hose heating connector that provides heating power to the heating wire.

10. The medical humidifier of claim 1,
wherein the housing further includes a display that displays the current temperature of the humidified air.
